(19) United States
(12) Patent Application Publication
(10) Pub. No.: US 2005/0100979 A1
(43) Pub. Date: May 12, 2005

(54) Methods for detecting oxidative stress

(76) Inventor: John Henry Thomas Power, Stirling (AU)

Correspondence Address: BIRCH STEWART KOLASCH & BIRCH, PO BOX 747, FALLS CHURCH, VA, 22040-0747, US

(21) Appl. No.: 10/651,056

(22) Filed: Aug. 29, 2003

(30) Foreign Application Priority Data
Sep. 30, 2002 (AU) 2002951775

(51) Int. Cl. C12Q 1/26

(52) U.S. Cl. 435/25

(57) ABSTRACT

The present invention provides a method for diagnosing a disease state associated with oxidative stress in a subject. The method includes the step of measuring the level of non-selenium glutathione peroxidase protein in a biological fluid or tissue obtained from the subject over time to detect an increase in the level of non-selenium glutathione peroxidase protein in the subject and/or measuring the level of non-selenium glutathione peroxidase protein in a biological fluid or tissue obtained from the subject and comparing the measured level of non-selenium glutathione peroxidase protein with a control level.

### FIELD OF THE INVENTION

The present invention relates to assays that can be used to detect oxidative stress in a subject and also diagnose disease states that are associated with oxidative stress. The assays may be useful in the detection of neurodengenerative diseases such as Parkinson's disease, Alzheimer's disease and Dementia.

### BACKGROUND OF THE INVENTION

Oxidative stress is a general term that is used to describe a state of cellular damage that is caused by reactive oxygen species (‘ROS’). ROS include free radicals and peroxides that can damage a specific molecule or an entire organism.

ROS are known to cause cell degeneration, especially in the brain. ROS have been implicated as a cause of neurodegenerative diseases such as Alzheimer's disease (AD), Dementia, Lou Gehrig's disease, Parkinson's disease (PD) and Huntington's disease.

It has also been postulated that subjects afflicted with cancer, heart disease or neurodegenerative disease are under severe oxidative stress for long periods of time before these illnesses become evident.

It is estimated that in the United States alone there are approximately 1 million PD sufferers and four million AD sufferers. Unfortunately, there is no definitive biological marker or behavior test to diagnose PD, AD or Dementia with Lewy bodies (DLB). As a result, misdiagnosis rates are as high as 20%. Utilising current methods, by the time a diagnosis is made a patient is likely to have incurred significant neuronal damage.

There is therefore a need for a diagnostic method that can be used in early diagnosis of neurodegenerative diseases or can be used to monitor disease progression and the effectiveness of any therapeutic intervention. Moreover, there is a need for a diagnostic method that is relatively simple to carry out, such as a simple blood test measurement based on a suitable biological marker. Biological markers have the advantage of identifying at risk subjects, early diagnosis, monitoring disease progression and improved accuracy. However, no such biological marker is currently available for PD, AD and DLB.

### SUMMARY OF THE INVENTION

The present invention arises out of the discovery of elevated levels of non-selenium glutathione peroxidase (NSGP) enzyme in the brains of subjects afflicted with AD, PD or DLB relative to the brains of non-afflicted subjects. It is postulated that the increased levels of NSGP reflects an increase in neuronal oxidative stress.

Accordingly, the present invention provides a method for detecting an increase in oxidative stress in a subject, the method including the step of:
monitoring the level of NSGP in a biological fluid or tissue obtained from the subject over time to detect an increase in the level of NSGP in the subject, and/or
measuring the level of NSGP in a biological fluid or tissue obtained from the subject and comparing the measured level of NSGP with a control level.

The present invention also provides a method for diagnosing a disease state associated with oxidative stress in a subject, the method including the steps of:
measuring the level of NSGP in a biological fluid or tissue obtained from the subject, and
comparing the measured level of NSGP with a control level.

The present inventors have developed oligopeptide fragments of NSGP and antibodies raised against these fragments were found to be specific for NSGP.

Therefore, the present invention also provides an isolated oligopeptide wherein antibodies raised against the oligopeptide are specific for NSGP. In one preferred form of the invention the oligopeptide contains the following amino acid sequence: RIRFHDFLGDSWGILFSHPR [SEQ ID NO:1]. In another preferred form of the invention the oligopeptide contains the following amino acid sequence: KKLFPKGVFTKELPSGKKYLR [SEQ ID NO:2].

The invention also provides an immunogenic conjugate that includes an oligopeptide of the present invention, and a carrier protein. The immunogenic conjugate may be used to raise antibodies that are specific for NSGP.

Therefore, the invention also provides an antibody that binds to an oligopeptide that includes the sequence of SEQ ID NO:1 or a fragment of SEQ ID NO:1, or the sequence of SEQ ID NO:2 or a fragment of SEQ ID NO:2.

The invention also provides an antibody that is specific for NSGP wherein the antibody is raised against an oligopeptide that includes the sequence of SEQ ID NO:1 or a fragment of SEQ ID NO:1, or the sequence of SEQ ID NO:2 or a fragment of SEQ ID NO:2.

In addition, the invention provides a method for producing antibodies that are specific for NSGP, the method including the steps of administering the immunogenic conjugate of the present invention to an animal, and collecting antibodies raised against the immunogenic conjugate.

The present invention also provides a method for inhibiting or alleviating one or more of the symptoms of a neurodegenerative disease associated with oxidative stress in a subject, the method including the step of up-regulating expression of NSGP in the subject.

As used herein the term ‘up-regulation’ in reference to expression of NSGP means that the protein is expressed at levels above the levels at which the protein is normally expressed in a subject that is not afflicted with the neurodegenerative disease. Whether or not expression of a protein is up-regulated can be determined by comparative immunohistochemical and western blot analysis of test subjects and appropriate control subjects.

‘Subject’ as used herein includes any mammalian single subject for which therapy is desired, including (but not limited to) humans, cattle, dogs, guinea pigs, rabbits, pigs, horses, or chickens. Most preferably, the subject is a human.

The method of the present invention may be used for prophylactic treatment and/or it may be used to maintain a condition or prevent further degeneration.

Alternatively, one or more of the symptoms of a neurodegenerative disease associated with oxidative stress in a subject may be inhibited or ameliorated by administering to the subject a mimic of NSGP. Preferably, the mimic is able to cross the blood brain barrier so that it is able to metabolise ROS in neurons.

The invention also provides a pharmaceutical composition for inhibiting or alleviating one or more of the symptoms of a neurodegenerative disease associated with oxidative stress in a subject, the composition including a substance that up-regulates expression of NSGP in the subject, and/or a substance that mimics NSGP. The pharmaceutical composition may contain suitable carriers and additives as is known in the art.

Throughout this specification reference is made to peptide, oligopeptide or protein sequences and by way of a shorthand notation the three and one letter abbreviations for amino acid residues in Table 1 are used in the specification.

TABLE 1

| | Three letter | One letter |
|---|---|---|
| Amino Acid | Abbreviation | Abbreviation |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### GENERAL DESCRIPTION OF THE INVENTION

Using the methods of the present invention it is possible to detect an increase in oxidative stress in a subject. This can be done by either monitoring the level of NSGP in a biological fluid or tissue obtained from the subject over time and/or measuring the level of NSGP in a biological fluid or tissue obtained from the subject and comparing the measured level of NSGP with a control level.

A number of disease states have either been shown or have been postulated to be associated with oxidative stress. These include neurodegenerative diseases, heart disease and cancer. Consequently, the method of the present invention may be used to diagnose any one or more of these disease states in a subject. It has previously been shown that subjects can be under severe oxidative stress for long periods of time before these illnesses become evident, and therefore the method of the present invention may be used in the early diagnosis of any one or more of these disease states.

The method of the invention may be particularly suitable for the diagnosis or therapeutic monitoring of neurodegenerative disease, in particular in PD, AD or Dementia patients, or for the early diagnosis of neurodegenerative disease, in particular PD, AD or Dementia.

According to the present invention a method for diagnosing a disease state associated with oxidative stress in a subject includes the steps of measuring the level of NSGP in a biological fluid or tissue obtained from the subject and comparing the measured level of NSGP with the level from a non-diseased subject.

The present inventors have developed antigenic fragments of NSGP and antibodies raised against either of these fragments were found to be specific for NSGP.

Therefore, the present invention also provides an isolated oligopeptide fragment of NSGP. In one preferred form of the invention the fragment contains the following sequence; RIRFHDFLGDSWGILFSHPR [SEQ ID NO:1]. In another preferred form of the invention the fragment contains the following sequence: KKLFPKGVFTKELPSGKKYLR [SEQ ID NO:2]. Alternatively, oligopeptides derived from other regions of the NSGP sequence may be prepared. The published amino acid sequence (see Kim, T. S. et al., 1997 “Identification of a human cDNA clone for lysosomal type Ca2+-independent phospholipase A2 and properties of the expressed protein” J. Biol. Chem. 272 (4), 2542-2550, and/or GENBANK ACCESSION #: D14662) of the NSGP protein is:

[SEQ ID NO:3]

MPGGL LLGDV APNFE ANTTV GRIRF HDFLG DSWGI LFSHP
RDFTP VCTTE LGRAA KLAPE FAKRN VKLIA LSIDS VEDHL
AWSKD INAYN CEEPT EKLPF PIIDD RNREL AILLG MLDPA
EKDEK GMPVT ARVVF VFGPD KKLKL SILYP ATTGR NFDEI
LRWIS LOLTA EKRVA TPVDW KDGDS VMVLP TIPEE EAKKL
FPKGV FTKEL PSGKK YLRYT PQP.

Oligopeptide sequences of about 10 or more amino acids may be suitable for raising antibodies that are specific for NSGP.

The amino acid sequence of alternative oligopepetides may be discovered by the use of overlapping or random oligopeptide generation or alternative computer simulation software so long as the alternatives permit the induction of antibodies of the same specificity as the chosen sequence, that is to say antibodies specific for NSGP.

The oligopeptide may also be homologous to any of the abovementioned oligopeptides provided that the oligopeptide provides antibodies that are specific for NSGP. In this context, an oligopeptide is considered homologous to an oligopeptide of the present invention when it is Immuno cross-reactive with antibodies specific for NSGP. It will be recognised by those skilled in the art that some amino acid sequences within the oligopeptide can be varied without significant effect on the structure or function of the oligopeptide. Thus for instance it is anticipated that ‘type’ amino acid substitutions still retain immuno-cross reactivity and as such a neutral amino acid may be conservatively substituted with another neutral natural or non-natural amino acid, an acidic amino acid may be conservatively substituted with a natural or non-natural acidic amino acid, a hydrophilic amino acid may be substituted with another hydrophilic amino acid, and so on, provided that the immunological function of the oligopeptide is not altered by the substitution.

Typically seen as conservative substitutions are the replacement of one for another among the aliphatic amino acids Ala, Val, Leu and Ile; interchange of the hydroxyl residues Ser and Thr; exchange of the acidic residues Asp and Glu; substitutions between the amide residues Asn and Gin; exchange of the basic residues Lys and Arg; and replacements among the aromatic residues Phe and Tyr. Preferably the homologous oligopeptide shares 50% homology with an oligopeptide of the present invention, more preferably shares 70% homology, and most preferably shares 90% homology.

The invention also extends to variants of these oligopeptides that are either shorter by a few amino acids, at the N-terminal or C-terminal end or both, or longer by a few amino acids. The variants may be obtained by chemical synthesis or by enzymatic digestion of NSGP.

To raise NSGP specific antibodies, one or more of the oligopeptides of the present invention can be conjugated as haptens to a suitable carrier protein to form an immunoconjugate. Suitable carrier proteins include diphtheria toxin, ovalbumin (OVA), bovine serum albumin (BSA) and keyhole limpet hemocyanin (KLH). The antibody may be produced by immunising a suitable animal such as mice, guinea pigs, rabbits, goats, sheep, horses, with one or more immunogenic conjugates of the present invention and isolating antibodies from the immunised animal. More specifically, when the antibody reaches sufficient titre, the animals can bled out and the specific antibody purified using a NSGP affinity column. The serum can be collected and the antibody titre evaluated, in a first stage, in a homologous system, against the oligopeptides used as immunogens, then against NSGP in order to select the antibodies which can be potentially used in the diagnostic test.

The present invention also relates to isolated antibodies whose production results from the use of the immunogenic conjugates defined above. These antibodies may be purified by affinity chromatography on a Sepharose™ type gel grafted with the same oligopeptide which served as hapten in the immunogenic conjugate, so as to select antibodies having the desired specificity. This can be controlled by the same immunoenzymatic assays which were used to choose the oligopeptide and which show that the purified antibodies specifically recognize naturally occurring NSGP.

The present invention also extends to monoclonal antibodies exhibiting the same specificity, that is for NSGP, which are induced according to conventional procedures using as the immunogen, the oligopeptides according to the present invention or derivatives thereof, and which are purified according to conventional methods.

For preparation of monoclonal antibodies, any technique which provides antibodies produced by continuous cell line cultures can be used. Examples include the hybridoma technique, the trioma technique, the human B-cell hybridoma technique. In a preferred method, the antibody producing cells may be fused with a myeloma cell to produce a pool of hybridoma cells which can then be screened for cells that produce the monoclonal antibody.

Monoclonal antibody fragments may also be used in the above method. Thus, the NSGP containing biological sample may be contacted with a fragment of a monoclonal antibody specific for an oligopeptide of the present invention.

Alternatively, additional antibodies capable of binding to NSGP may be produced in a two step procedure through the use of anti-idiotypic antibodies. Such a method makes use of the fact that antibodies are themselves antigens, and that, therefore, it is possible to obtain an antibody which binds to a second antibody. Thus, NSGP specific antibodies can be used to immunise an animal and the splenocytes of the animal are then used to produce hybridoma cells, and the hybridoma cells screened to identify clones which produce an antibody whose ability to block the NSGP specific antibody can be blocked by the NSGP protein. Such antibodies comprise anti-idiotypic antibodies to the NSGP protein specific antibody.

Antibodies capable of binding to NSGP may also be prepared by raising antibodies to any class of NSGP, including NSGP from rat olfactory mucosa (rat), bovine ciliary body epithelium (bovine), mouse skin, liver and kidney (mouse) as well as porcine, ovine, ave (chicken, duck), rabbit or guinea pig sources. It is thought that the sequence of NSGP is highly conserved (>90% homology) among these species.

Reference to an antibody throughout this specification should also be taken to include a fragment of a monoclonal antibody. Therefore the term includes, but is not limited to, Fab, Fv and peptide fragments of the monoclonal antibody, and it may also include such fragments when made as part of a different larger peptide or protein, which may be the product of a recombinant vector. Thus the variable region of the respective monoclonal antibody may be cloned and be made part of a hybrid protein.

The biological fluid that is assayed may be any biological fluid in the subject that is capable of containing NSGP including, but not limited to, serum, plasma, whole blood, cerebro spinal fluid, amniotic fluid, and synovial fluid.

The tissue that is assayed may be any issue of the subject that is capable of containing NSGP including, but not limited to, brain issue.

The levels of NSGP may be measured using an agent that specifically recognises the NSGP protein. More specifically, the levels may be measured by detecting the binding of NSGP with an antibody specific for NSGP. Alternatively, the levels may be measured by detecting the binding of NSGP with a labelled substrate for NSGP.

In one specific form, the invention provides a method for detecting oxidative stress in a subject, the method including the steps of:
producing antibodies specific to at least one peptide fragment of NSGP, or derivative thereof,
obtaining a putative NSGP containing biological sample from the subject,
contacting the biological sample with the antibodies under conditions for formation of an antibody:NSGP complex, and
assaying for the formation of the antibody:NSGP complex to detect the presence and/or levels of NSGP.

In one form of the invention the step of assaying for the formation of antibody:NSGP complex involves detecting the complex using a second revealing antibody. This method is commonly called ‘sandwich immunoassay’.

The revealing antibody could be a second anti-peptide antibody derived from an NSGP protein sequence or alternatively a polyclonal antibody derived from native NSGP.

This method therefore comprises the steps of binding specific anti-peptide antibodies to a support; immunocapture of NSGP in a sample of biological material by the antibodies; and revealing of the immunocaptured NSGP by a second labelled anti-peptide antibody. Other embodiments of the same immunocapture assay principle can also be envisaged and form part of the present invention. According to a preferred embodiment of the method, one of the anti-peptide antibodies is bound to a support. The antibodies thus bound permit the specific immunocapture of the NSGP present in the sample to be assayed, the latter being preferably serially diluted. The NSGP thus captured by the anti-peptide antibody is revealed with the aid of a second antibody which is labelled in order to increase the sensitivity of the detection. The second type of antibody or revealing antibody is obtained after immunization of animals with one or the other of the purified oligopeptides; these antibodies are purified by utilizing NSGP conjugated Sepharose beads on a column; they are then labelled. The second type of antibody may optionally be a monoclonal antibody or a polyclonal antibody raised against native NSGP. The NSGP titre of the sample to be assayed is determined by comparison with a reference curve established with a standard sample of purified NSGP. Alternatively, the second antibody may remain unlabelled and the use of a third labelled antibody, with specific reactivity against the second antibody may be utilized for the purposes of detection.

In contacting the biological sample with the antibody, the antibody may be attached or conjugated to a carrier molecule or attached or conjugated to a solid support. A solid support in the present invention means any solid material to which the antibody can be complexed or attached. Examples of such solid supports include, but are not limited to, microtitre plates, petri dishes, bottles, slides, and other such containers made of plastic, glass, polyvinyl, polystyrene, and other solid materials which allow detection of labelled antibodies. Other suitable carriers for binding the antibody exist or will be able to ascertained by routine experimentation.

In a preferred form of the invention the antibody is covalently or noncovalently bound to the surface of a microtitre well. A serum sample suspected of containing NSGP may then be added, unbound sample washed away and the level of NSGP bound in the antibody:NSGP complex assayed. However, to determine levels of NSGP in a sample, it may be preferable to serially dilute the antibody in several wells in a microtitre plate and to contact a homogeneous biological sample with each of the wells.

In another preferred form of the invention the antibody is used as a target in a microarray which can be used to monitor the expression level of NSGP in a sample. One suitable method of determining the presence of NSGP by microarrays is as follows. NSGP specific antibodies are immobilised in a specific and ordered array onto a solid plate (typically a glass plate). Then the plate is exposed to one or several targets either separately or in a mixture. In this way the amount and type of each target can be determined by measuring the location and concentration of each marker molecule at each test spot on the microarray. To eliminate sample variation, the signal ratio between two competing samples is the preferred measurement. Microarrays may be prepared, used, and analyzed using methods known in the art.

The assay for the formation of antibody:NSGP complex preferably involves adding a compound that enables detection of the NSGP which is specifically bound to the antibody.

In a preferred form of the invention the assay is an ELISA and the raised antibodies may be used to bind the NSGP in the sample, and a labelled antibody specific for another immuno-recognition site on NSGP can be used to assay for bound NSGP. These assays employ a wide variety of labels including radionuclides, enzymes, fluorescers, chemiluminescers, particles, ligands, enzyme substrates, enzyme cofactors, enzyme inhibitors, light emitter-quencher combinations.

It will be appreciated that the antibodies being assayed may be members of any of the five major classes of antibodies and therefore the diagnostic method encompasses IgA, IgD, IgE, IgG and IgM antibodies and the labelled antibodies for use in an ELISA may be IgA, IgD, IgE, IgG, and IgM antiantibodies, respectively.

The labelled antibodies specific for NSGP may be labelled with a radioisotope, which can then be determined by such means as the use of a gamma counter or a scintillation counter.

Another way in which the antibodies specific for NSGP can be detectably labelled is by linking to an enzyme. This enzyme, in turn, when later exposed to its substrate, will react with the substrate in such a manner as to produce a chemical moiety which can be detected, for example, by spectrophotometric, fluorometric or visual means. Examples of enzymes which can be used to detectably label the antibody include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase. Avidin-biotin binding may be used to facilitate the enzyme labelling.

It is also possible to label the antibodies specific for NSGP with a fluorescent compound. When the fluorescently labelled antibody is exposed to light of the proper wavelength, its presence can then be detected due to the fluorescence of the dye. Among the most commonly used fluorescent labelling compounds are fluorescein isothiocyanate, rhodamine, phycoerytherin, phycocyanin, allophycocyanin, o-phthaldehyde and fluorescamine.

The antibodies specific for NSGP can also be detectably labelled using fluorescent emitting metals such as 152Eu, or others of the lanthanide series. These metals can be attached to the antibody molecule using such metal chelating groups as diethylenetriaminepentaacetic acid (DTPA) or ethylenediaminetetraacetic acid (EDTA).

The antibodies specific NSGP can also be detectably labelled by coupling them to a chemiluminescent compound. The presence of the chemiluminescent-tagged antibody is then determined by detecting the presence of luminescence that arises during the course of a chemical reaction. Examples of particularly useful chemiluminescent labelling compounds are luminol, isoluminol, theromatic acridinium ester, imidazole, acridinium salt and oxalate ester.

Likewise, a bioluminescent compound may be used to label the antibodies specific for NSGP. Bioluminescence is a type of chemiluminescence found in biological systems in which a catalytic protein increases the efficiency of the chemiluminescent reaction. The presence of a bioluminescent antibody is determined by detecting the presence of luminescence. Important bioluminescent compounds for purposes of labelling are luciferin, luciferase and aequorin.

Another technique which may also result in greater sensitivity when used in conjunction with the present invention consists of coupling the antibodies specific for NSGP to low molecular weight haptens. The haptens can then be specifically detected by means of a second reaction. For example, it is common to use haptens such as biotin (reacting with avidin) or dinitrophenyl, pyridoxal and fluorescamine (reacting with specific anti-hapten antibodies) in this manner. The step of assaying for formation of the antibody:NSGP complex may include the step of separating the bound antibody:NSGP complex from unbound NSGP. Thus, the raised antibodies, or fragments thereof may be labelled as discussed previously. Any antibody:NSGP complex formed by contacting the sample with the antibody may be separated from unbound antibody using suitable techniques such as immunoprecipitation or techniques for separation based on size. For example, a mixture obtained after contacting the sample with antibody may be filtered through a suitable membrane so that antibody; NSGP complex is retained on the membrane and unbound antibody passes through the membrane. The labelled antibody:NSGP complex can then be quantitatively assayed using standard techniques for the label used.

Other known methods may also be used to assay for levels of NSGP in the biological sample. For example NSGP expression in tissues can be studied with classical immunohistological methods. In these, specific recognition is provided by the primary antibody but the secondary detection systems can utilise fluorescent, enzyme, or other conjugated secondary antibodies. As a result an immunological staining of tissue section for pathological examination is obtained. Tissues can also be extracted, for example with urea and neutral detergent, for the liberation of the NSGP protein for Western-blot or dot/slot assay.

The present invention also relates to a ready-for-use kit or set for the implementation of the assay method described above. This kit comprises: a titration plate, divisible or otherwise, preferably with 96 wells, in which the anti-peptide antibody has been bound, according to a conventional method, and covers the entire surface of the wells, a solution for diluting the samples to be assayed, preferably consisting of a buffered solution (Tris or phosphate), NaCl, protein (casein, ovalbumin or serum albumin and the like) at a concentration of 0.1 to 1%, detergent and preserving agent (sodium azide or merthiolate) and a washing solution of the same composition but without proteins, a standard consisting of recombinant, purified NSGP, a solution of labeled anti-NSGP oligopeptide revealing antibodies; these antibodies may be polyclonal or monoclonal and result from an immunization, according to a conventional procedure; these antibodies are labeled either with biotin, or with a revealing enzyme (such as alkaline phosphatase or horseradish peroxidase). The antibody solution is prepared in Tris or phosphate buffer containing 150 mM NaCl, 0.1 to 1% of overload protein (serum albumin, ovalbumin or casein), glycerol and a preserving agent, a substrate for revealing the antibody labelling, such as for example pNPP (4-nitrophenylphosphate) for alkaline phosphatase, ortho-phenylenediamine for peroxidase and so on.

The kit according to the invention can be used for any assay of NSGP in biological fluids or tissue extracts, in particular for the diagnosis and therapeutic monitoring neurodegenerative diseases including PD, AD and Dementia.

The oligopeptides of the present invention may also be used to directly or indirectly detect the presence of antibodies to NSGP present in the subject.

The present invention also provides a method for inhibiting or alleviating one or more of the symptoms of a neurodegenerative disease associated with oxidative stress in a subject, the method including the step of up-regulating expression of NSGP in the subject.

As used herein the term ‘up-regulatlon’ in reference to expression of NSGP means that the protein is expressed at levels above the levels at which the protein is normally expressed in a subject that is not afflicted with the neurodegenerative disease. Whether or not expression of a protein is up-regulated can be determined by comparative immunohistochemical and western blot analysis of test subjects and appropriate control subjects.

‘Subject’ as used herein includes any mammalian single subject, including (but not limited to) humans, cattle, dogs, guinea pigs, rabbits, pigs, horses, or chickens. Most preferably, the subject is a human.

The method of the present invention may be used for prophylactic treatment and/or it may be used to maintain a condition or prevent further neuron degeneration.

In an alternative form of the invention, one or more of the symptoms of a neurodegenerative disease associated with oxidative stress in a subject may be inhibited or ameliorated by administering to the subject a mimic of NSGP. Preferably, the mimic is able to cross the blood brain barrier so that it is able to metabolise ROS in neurons.

The invention also provides a pharmaceutical composition for inhibiting or alleviating one or more of the symptoms of a neurodegenerative disease associated with oxidative stress in a subject, the composition including a substance that up-regulates expression of NSGP in the subject, and/or a substance that mimics NSGP.

The pharmaceutical compositions of this invention can be administered to humans and other animals orally, rectally, parenterally (i.e. intravenously, intramuscularly, or sub-cutaneously), intracisternally, intravaginally, intraperitoneally, topically (as by powders, ointments, or drops), transdermally, bucally, or as an oral or nasal spray. Multiple administration may be required.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, or dispersing agents. For example, it may be desirable to administer the composition together with an adjuvant such as Freund's (complete or incomplete), mineral gels, surface active substances such as peptides or oil emulsions. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If desired, and for more effective distribution, the compounds can be incorporated into slow release or targeted delivery systems such as polymer matrices, liposomes, and microspheres.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite day, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

The composition may be administered at any time. Preferably the composition is administered at or after the time of diagnosis.

Formulations containing the composition of the invention may conveniently be presented in unit-dose or multi-dose containers, e.g. sealed ampoules and vials. Preferred unit dosage formulations are those containing a daily dose or unit, daily sub-dose, or an appropriate fraction of the administered ingredient.

It is contemplated that an attending clinician will determine, in his or her judgement, an appropriate dosage and regimen, based on the subjects age and condition as well as the severity of the neurodegenerative disease.

### DESCRIPTION OF THE FIGURES

FIG. 1(A) shows the results of PAGE analysis of soluble brain proteins from white and gray matter of normal, PD, and DLB brains. Lane 1, PD FC and GM; Lane 2 PD, FC, and WM; Lane 3, PD and SN; Lane 4, NSGP recombinant protein; Lane 5, DLB, FC and GN; Lane 6, DLB, FC, and WM; Lane 7, N, FC, and GM; Lane 8, N, FC and WM; and Lane 9, N and SN. Gel was stained with Coomassie blue. Molecular weights in kd are indicated at the left. Abbreviations: FC, frontal cortex; GM, gray matter, WM, white matter; SN, substantia nigra; DLB, dementia with LBs; N, normal.
(B) shows a Western blot of the same samples as indicted in (A). Primary antibody was protein A-purified rabbit anti-NSGP IgC, secondary antibody was a biotin-labeled donkey anti-rabbit (711-065-152, Jackson ImmunoResearch) and visualized with a Vector ABC kit using DAB.

FIG. 2(A-D) shows low-power images (original magnifications ×400) of the NSGP immunocytochemistry of gray and white gray matter from normal (A and B) and DLB (C and D) brain tissue. NSGP was visualized using a biotinylated donkey and anti-rabbit secondary antibody and a Vector ABC staining kit with DAB. Sections were lightly counterstained with hematoxylin. NGSP-positive glial cells were markedly increased in both gray and white matter of DLB compared to normal. The arrows in C and D indicate NSGP-positive glial cells. (E-G): Comparison of astrocyte labeling (oil immersion; original magnifications ×1000) with NSGP antibodies in normal white cortical matter (E) and white (F) and gray matter (G) of DLB cortical tissue. (H-I): LB labeling with rabbit anti-NSGP antibodies (H) and labeling with sheep ant-α-synuclein antibodies (I) within the gray matter of DLB tissue (oil immersion; original magnifications, ×1000). Immunoreactivity was visualized with biotinylated secondary antibodies and a Vector ANB kit using DAB. Arrows indicate the positively stained LBs within the cytoplasm of neurons. (J-R): J to L show α-synuclein

FIG. 3 shows the number of NSGP-positive glial cells in the white and gray matter of control (n=7), DLB (n=6), and Pd (n=4) brains. Mean ±SE. Significance was determined using unpaired Student's t-tests and the asterisks indicate significance at the 0.05 level of probability.

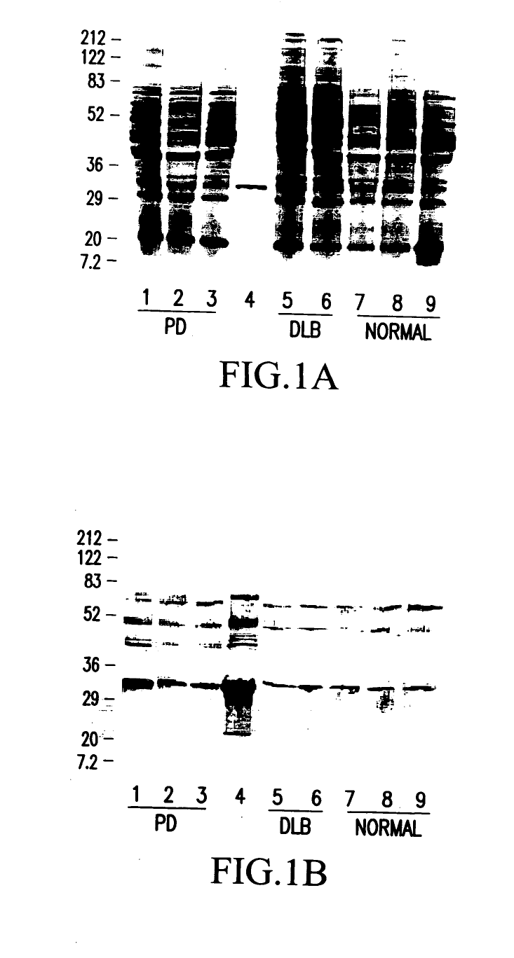

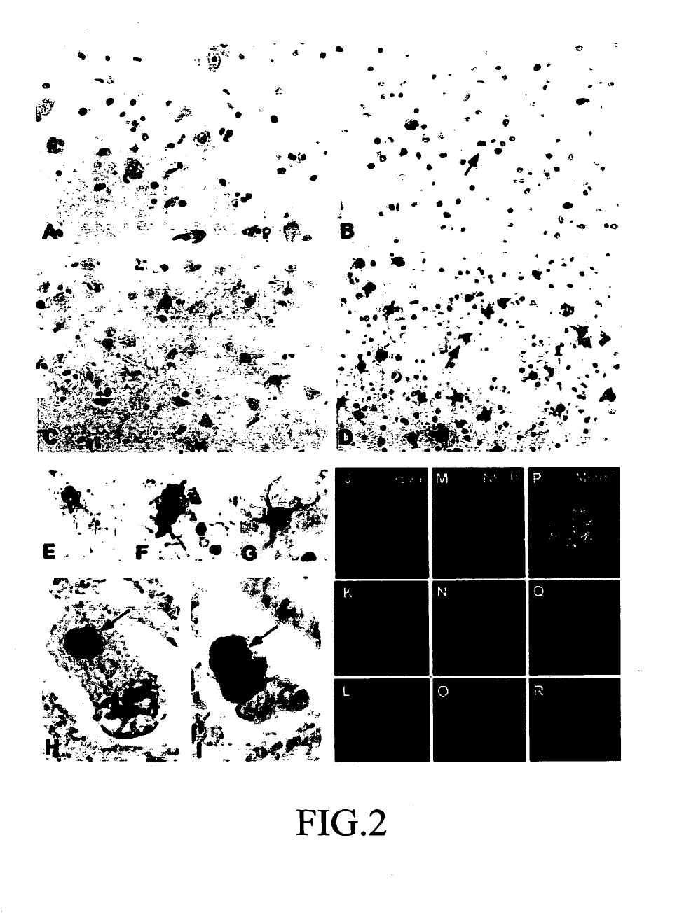

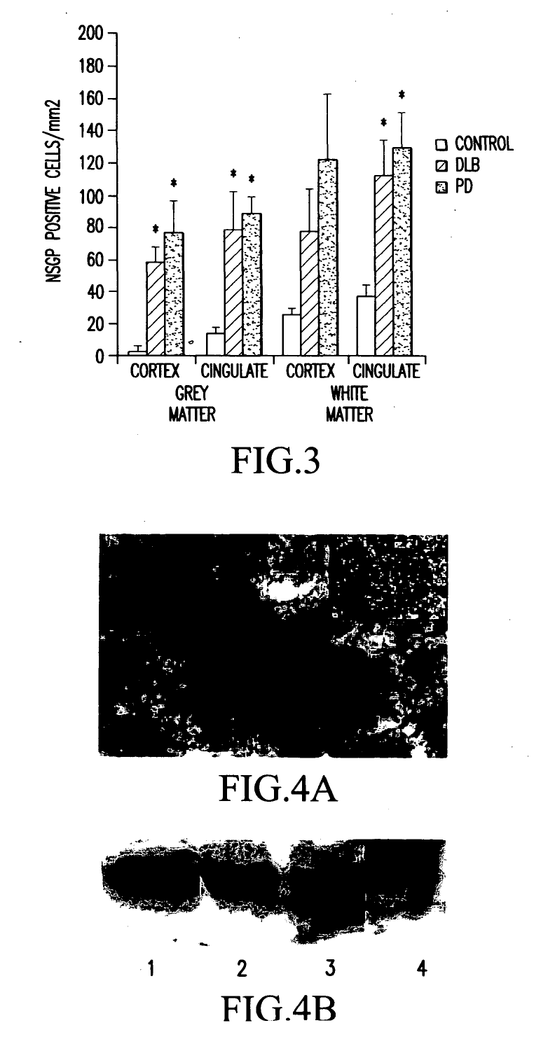

FIG. 4(A) shows electronmicograph of a LB purified using magnetic beads and immunostained with a NSGP primary antibody and visualized with a secondary antibody conjugated to 12-nm gold. Top right inset shows the intact LB bound to magnetic beads (MB), small arrows indicate the boundary of LB. The area indicated was taken at higher power and shows the distribution of the gold particles.
(B) immunoprecipitation of NSGP by anti-α-synuclein antibodies from a cytosol fraction of normal control human brain. Immuno-blotting was conducted using chicken IgY raised against recombinant NSGP, followed by blotinylated donkey anti-chicken IgY, and streptavidin-biotin-HRP complex. The membrane wss developed using X-ray film and a enhanced chemilumescence kit (Amersham Pharmacle Biotec, Sydney, Australia). Lane 1, recombinant NSGP (˜15 ng.) Lane 2, Precipitates using antibody to α-synuclein C-terminal 116 to 131 amino acids. Lane 3, Precipitates using antibody to α-synuclein N-terminal 11 to 26 amino acids. Lane 4, Precipitates from acetone-extracted cytosol using antibody to α-synuclein N-terminal 11 to 26 amino acids.

### DESCRIPTION OF PREFERRED EMBODIMENTS OF THE INVENTION

The following description illustrates the specific aspects of the development of the invention as well as embodiments thereof, without however limiting its scope.

One preferred embodiment of the invention is for a diagnostic method of detecting the protein enzyme NSGP in blood, serum, plasma or tissue samples in a body as a measure of the oxidative stress of the subject. The invention is useful, inter alia, in the early detection of a range of diseases, particularly neurodegenerative diseases including, but not limited to, Parkinson's disease, Alzheimer's disease and Dementia and for measuring disease progression and the effects of therapeutic intervention by virtue of the levels of the enzyme in the subject.

### EXAMPLE 1

Production of Recombinant NSGP

Screening of an adult rat lung cDNA library (Clonetech Laboratories, Palo Alto, Calif.) with rabbit polyclonal anti-NSGP (Power et al. Exp Lung Res 1999, 25:379-391) produced a 1435-bp cDNA. Sequencing of this clone demonstrated that it was identical to the published sequence of a rat lung acidic Ca²⁺-independent PLA₂ (Kim et al. Am J Physiol 1998, 274:L750-L761) that was later shown to possess both NSGP and phospholipase activity (Chen et al., J Biol Chem 2000, 275:28421-28427). Using this cDNA as a template, polymerase chain reaction was performed using primers 5′-GGCAATTCATGCCCGGAGGGCTGCTTCTC-3′ and 5′-CCGCTCGAGCGGGTTCCCGCAGACTTAAGGCTG-3′ which included restriction sites for EcoR1 and Xho1 (in bold) on the upstream and downstream primers, respectively. The amplicon generated by these primers spans the entire coding sequence of NGSP. After amplification, the products were treated with EcoR1 and Xho1 to produce cohesive ends, purified, and cloned in frame into the glutathione S-transferase expression vector pGEX-6P (Amersham Pharmacia Biotech, Piscataway, N.J., USA); the expression construct was sequenced to ensure fidelity of polymerase chain reaction amplification. Transformation of bacteria, purification of the fusion protein and cleavage of the glutathione S-transferase tail from the recombinant NGSP were all performed according to the manufacturer's instructions. The purified protein was concentrated by lyophilization and used for antibody production.

### EXAMPLE 2

Production of Antibodies Against NSGP

Antiserum was raised in New Zealand White rabbits using recombinant rat NSGP as antigen. The IgG fraction was obtained using Protein A affinity chromatography and concentrated using a Centriprep concentrator. The concentrated antibody was stored in phosphate-buffered saline (PBS) in small aliquots at −20° C. Chicken anti-NSGP antibodies were obtained from Antibody Technology Pty Ltd., Stirling South Australia, Australia.

### EXAMPLE 3

Production of Antibodies Against α-Synuclein

α-Synuclein antibodies were raised in sheep against human α-synuclein peptide sequence 116 to 131. The antibodies were affinity-purified using the antigen and extensively characterized as described (Gai et al. Exp Neurol 2000, 166:324-333; Braak et al. Neurosci Let 1999, 265:67-69; Gai et al. J Neurochem 1999, 73:2093-2100; Jenson at al. J Biol Chem 2000, 275:21500-21507).

### EXAMPLE 4

Production of Antibodies Against Glial Fibrillary Acidic Protein (GFAP)

GFAP Antibodies were Obtained from DAKO Pty., Botany, NSW, Australia.

### EXAMPLE 5

Assays for NSGP in Human Brain Tissue

The human brain used in this study is listed in Table 1. Brain tissue was obtained from the National Health and Medical Research Council South Australian Brain Bank. These brains were removed at autopsy, generally within 24 hours of death. Brains were bisected and one half was snap-frozen at −70° C. while the other half was either perfusion-fixed or immersion-fixed with 4% formaldehyde and 2% picric acid as previously described (Gai et al. Exp Neurol 2000, 166:324-333). Tissue blocks were embedded in paraffin and 5-μm sections obtained for each brain. Each case was examined from a clinicopathological perspective to determine the pathology. In this article brain tissue from seven PD cases, five DLB cases, and five normal cases were examined. Brain regions examined include the frontal cortex, cingulated, and the brain stem.

TABLE 1
Human Brain Tissues Used in Study

| Case | Age | Gender | Region |
|---|---|---|---|
| PD (123/93) | 65 | Male | BS |
| PD (52/99) | 84 | Male | BS |
| PD (7/95) | 68 | Female | BS |
| PD (P21) | 80 | Female | BS, C. MFC |
| PD (P22) | 85 | Female | C MFC |
| PD (P25) | 83 | Female | MFC |
| PD (P31) | 81 | Female | C, MFC |
| DLB (P23) | 67 | Male | C, MFC |
| DLB (P26) | 91 | Female | C, MFC |
| DLB (P30) | 81 | Female | C, MFC |
| DLB (P36) | 80 | Male | C, MFC |
| DLB (P40) | 86 | Male | C, MFC |
| Normal (N10) | 79 | Female | C, FC |
| Normal (N18) | 69 | Male | C, FC |
| Normal (N19) | 61 | Female | C, FC |
| Normal (N20) | 84 | Female | C, FC |
| Normal (N24) | 71 | Female | C, FC |

Abbreviations:
PD, Parkinson's Disease;
DLB dementia with Lewy bodies;
BS brain stem;
C, cigulate;
MFC, mid-frontal cortex;
FC, frontal cortex.

Homogenates

Frozen tissue from the cortex, cingulate, and substantia nigra from each case was carefully dissected to obtain white and gray matter and homogenized in PBS using a motorized Wheaton Teflon pestle tissue grinder (clearance, 0.15 to 0.23 mm). The homogenate was centrifuged at 8000×g for 30 minutes to remove particulate matter and the supernatant was assayed for total protein and frozen at −70° C.

LB Isolation

Cortical LBs were isolated using magnetic bead immunoprecipitation from fresh frozen cortices from DLB cases as described (Gai et al. Exp Neurol 2000, 166:324-333; Gai et al., J Neurochem 1999, 73:2093-2100). For immunoelectron microscopy, isolated LBs were pelleted in a microcentrifuge at 13,000×g for 5 minutes, fixed for 10 minutes with 0.25% glutaraldehyde-2% formaldehyde in PBS, pH 7.4, post-fixed in 0.5% osmium tetroxide for 30 minutes, and processed as described in the Electron microscopy section.

One Dimensional Electrophoresis and Western Blotting

Proteins from brain homogenates were separated using a Bio-Rad Minigel System (Bio-Rad, Richmond Calif.) and 4 to 20% precast polyacrylamide gel electrophoresis gels (Gradipore Ltd., French's Forrest, NSW. Australia). The separated proteins were transferred onto nitrocellulose using a Semidry Transfer Unit (model TE70 SemiPhor; Hoeffer Scientific Instruments, Pharmacia) using a transfer buffer of 0.25 mol/L Tris, 0.192 mol/L glycine, and 20% methanol for 90 minutes. The nitrocellulose membrane containing the transferred protein was blocked with milk proteins and incubated overnight with a primary antibody to NSGP. The antigen; antibody complex was visualized using a Vector ABC kit (Vector Laboratories Inc, Burlingame, Calif.).

Immunohistochemistry

Co-Localization of NSGP and α-Synuclein

Sections from each brain were incubated with either sheep anti-α-synuclein as a marker for LBs, rabbit anti-NSGP, or both antibodies for 18 hours. For 3,3′ diaminobenzidine tetrahydrochloride dihydrate (DAB) staining, the single antibodies were visualized with either donkey anti-sheep-conjugated horseradish peroxidase (Jackson ImmunoResearch) and the antibody complex was visualized using a Vector ABC kit. Sections were examined and photographed with an Olympus BX50 microscope linked to a Kodak EOS-DCS digital camera. For fluorescence staining the α-synuclein was visualized with donkey anti-sheep-conjugated fluorescein isothiocyanate (Jackson ImmunoResearch) and NSGP with donkey anti-rabbit-conjugated Cy5 (Jackson ImmunoResearch). Sections were examined using a Bio-Rad confocal laser scanning microscope and software package (Bi-Rad MRC 1024, Bio-Rad).

Immunoabsorption of Antibody

To confirm that the staining observed was specific, two aliquots of NSGP antibody as used in the previous staining were prepared, with one aliquot being immunoabsorbed for 24 hours with excess NSGP antigen. Both aliquots were then centrifuged and the supernatants used to stain identical tissue and Western blots.

Localization of NSGP and GFAP

Sections from each brain were incubated for 18 hours with antibodies to NSGP and GFAP to determine whether NSGP was specifically up-regulated in astrocytes. For fluorescence staining, NSGP was detected using donkey anti-rabbit-conjugated Cy5 (Jackson ImmunoResearch) and GFAP with donkey anti-mouse-conjugated Cy3 (Jackson ImmunoResearch). Sections were examined using a Bio-Rad confocal laser-scanning microscope and software package.

Electron Microscopy

Fresh brain pieces and pellets from magnetic bead immunoisolation were immersion-fixed with 2.0% paraformaldehyde, 0.35% glutaraldehyde (w/v) in 0.1 mol/L phosphate buffer at pH 7.4 for 3 hours and post-fixed in 0.5% osmium tetroxide (w/v) for 1 hour at 4° C. blocks were dehydrated through graded acetone at 4° C. and embedded in L. R. white Resin (London Resin, Basingstoke, UK). Ultra thin sections were incubated for 3 hours with rabbit anti-NSGP and visualized with donkey anti-rabbit-conjugated 12-nm gold (Jackson ImmunoResearch). NSGP preimmune serum was used as a negative control. The sections were stained with lead citrate and uranyl acetate and examined in a JEM 1200 EX electron microscope (Jeol, Tokyo, Japan).

Counting of Positive Glial Cells

NSGP-positive glial cells were counted at ×40 magnification with a graticule eyepiece (250 μm² or 0.0625 mm²) using an Olympus HO-2 microscope from five fields from seven control, six DLB, and four PD cases. In each case, the regions were selected from the white and gray matter of the frontal cortex and cingulated. The positive cells from the five randomly selected regions were averaged and divided by the area of the graticule (0.0625 mm²) and expressed as cells/mm². The mean and SE of the positive cells from each case are shown in FIG. 4. Significance at the 0.05 level of probability was determined between grouped data using Student's t-tests. The brain stem of another three PD cases were examined histologically but were not included here as the cingulated and cortex were not available.

Immunoprecipitation

Fresh-frozen cortical tissue from two cases without evidence of neurological disease were homogenized in 10 vol of PBS (pH 7.4) containing a cocktail of protease inhibitors, using a motorized Wheaton Teflon pestle tissue grinder. The homogenate was centrifuged at 8000×g for 30 minutes to obtain a cytosol fraction. In some experiments, the cytosol fraction was extracted to remove residue lipids. Affinity-purified sheep antiserum was raised against α-aynuclein C-terminal (116 to 131 amino acids) and affinity-purified rabbit antiserum against N-terminal (11 to 26 amino acids) was covalently coated to tosylactivated magnetic beads (dynabeads M-500) according to the manufacturers instructions. Coated beads were mixed with the human brain cytosol fraction (˜2×10⁷ beads/ml) and incubated overnight at 4° C. The beads were washed four times with PBS, and bound material was eluted with sodium dodecyl sulfate sample buffer. The sample was run on a one-dimensional PAGE gel and Western blotted as described above. The blot was probed with a chicken anti-nonselenium glutathione peroxidase antibody (Antibody Technology Pty Ltd).

### EXAMPLE 6

Identification and Preparation of Fragments of NSGP

The amino acid sequences of various human NSGP peptides was analysed using the PINSOFT software which makes it possible to define the hydrophilicity and flexibility profile of the primary sequence. The following oligopeptides were chosen and synthesized based on the known sequence of NSGP:

| | | |
|---|---|---|
| Oligopeptide 1: | RIRFHDFLGDSWGILFSHPR | [SEQ ID NO:1] |
| Oligopeptide 2: | KKLFPKGVFTKELPSGKKYLR | [SEQ ID NO:2] |

The oligopeptides can be synthesised using published procedures, with the sequences synthesised on a solid phase in the direction “C-terminal end” towards the “N-terminal end”. The purity of the amino acids used for the syntheses is always greater than or equal to 99% (HPLC purity). The different reagents and solvents Involved in the cutting step (hydrofluoric acid, ether, p-cresol and trifluoroacetic acid) are characterized by a purity of at least 99%. The purification of the peptides is performed and are analysed by high pressure liquid chromatography (HPLC).

### EXAMPLE 7

Preparation of Haptens

The oligopeptides can then be conjugated as haptens on diptheria toxin. The conjugates can be prepared by coupling the oligopeptides to a carrier protein so as to be used within the framework of a program of immunization of animals. The conjugate necessarily consists of a hapten represented by one of the oligopeptide sequences described above onto which there has generally been grafted, either at the N-, or C-terminal end, any amino acid (such as for example tyrosine). The additional residue is always incorporated into the oligopeptide chain during the peptide synthesis. This additional amino acid serves as a linkage between the sequence itself and a bifunctional reagent (such as bisdiazobenzidine, glutaraldehyde, m-maleimidobenzoyl-N-hydroxysuccinimide ester, carbodiimide) which is used to couple the oligopeptide structure to the carrier protein, diptheria toxin. The coupling is carried out according to standard procedures.

### EXAMPLE 8

Generation of NSGP-Specific Antibodies

The diptheria toxin-bound oligopeptides can be used to immunize rabbits or sheep, according to a conventional procedure: A mass of conjugate corresponding to 100 μg of peptide (rabbit) or 1 mg (sheep) is used per injection. The injection is performed intradermally and repeated 3 times at 3 to 4 week intervals. The serum is collected and the response with respect to antibodies directed against the oligopeptide (“anti-peptide antibodies”) is measured using a standard ELISA-based technique.

An assay based on competition between the recognition of NSGP insolubilized onto a microtitre plate and recognition of NSGP in solution, by the anti-peptide antibodies makes it possible to verify whether the antibodies are capable of binding to the protein in solution. In order to selectively recover antibodies specific for NSGP, the sera recovered can be subjected to a purification by affinity chromatography on a gel grafted with NSGP which serves as immunogen, by grafting of 5 mg of NSGP onto 1 ml of high-performance Sepharose gel (Pharmacia) preactivated by means of N-hydroxysuccinimide (NHS-activated HiTrap column). Deactivation of the active groups not coupled to the ligand and washing of the non-specifically bound ligands using a 0.5M saline solution of ethanolamine pH 8.3 and a 0.1M saline solution of acetate pH 4.0. These solutions both contain 0.5M NaCl and are used alternately over cycles of 6 ml of each solution at each step (three cycles). Then equilibrate the column with a PBS solution before loading the serum. The loading of about 10 ml of rabbit/sheep serum onto the column previously equilibrated with PBS buffer (phosphate-buffered saline) is then followed by washing the column with PBS buffer and elution of the specific anti-peptide antibodies by passing 0.1M citrate buffer pH 2.

The antibodies can then be concentrated on an ultrafiltration membrane (10 to 30,000 cut-off). The antibodies thus purified specifically recognize human NSGP. These antibodies are capable of recognizing the human NSGP bound to an insoluble support or in solution in a liquid phase.

For titration of an antibody solution, the titration method can be applied to sera collected during the selection of the peptides as well as to the specific purified antibody. The method is based on the recognition of antigen insolubilised on a microtitre plate and the revealing of the antibody bound by a labelled anti-antibody. 96-well microplates, conventionally used for titrations in various fields, are preferably used. The titration is carried out according to the following procedure: Sensitize the wells of the microplate with 100 μl of antigen solution (oligopeptide or purified NSGP) at 2 or 1 μg/ml respectively (sensitization buffer 10 mM Tris-HCl, pH 8.5, 100 mM NaCl) and then incubated overnight at 4° C. or for 2 hours at 37° C. Empty the wells and saturate the non-specific binding sites with a solution of protein such as for example 0.5% gelatine prepared with 50 mM Tris-HCl buffer, pH 7.8, 150 mM NaCl for 30 minutes at room temperature. Empty then wash the wells 3 times with 50 mM Tris-HCl solution at pH 7.8 containing 150 mM NaCl and 0.1% TWEEN 20. After removal of the last washing solution, distribute 100 μl of dilution buffer (50 mM Tris-HCl buffer at pH 7.8 containing 150 mM NaCl, 0.1% TWEEN 20 and 0.5% gelatine) into all the wells except the first well of each line.

Each of the unused wells is reserved for the deposition of each of the samples (serum or purified antibody) to be tested and of the control serum (negative control). These samples should often be diluted 50 to 200 fold with the dilution buffer. The volume of deposit is 200 μl in all cases. The dilutions (2-fold serial dilution) are performed from well to well by transferring 100 μl of solution. The transfer starts with the well containing the 200 μl of serum sample to be tested. Make provisions to reserve at least one well for the internal zero of the test, which is performed at the time of the optical density readings: this well should not be used to dilute any serum and should therefore contain only the dilution buffer. The 100 μl removed from the last well are eliminated and then incubate for 2 hours at room temperature. Empty the plate and wash the wells with a series of 5 successive washes. Eliminate the last washing solution and then distribute 100 μl of revealing solution, that is to say an anti-antibody conjugate labelled (for example with alkaline phosphatase). This anti-antibody is specific for the species from which the serum is obtained (rabbit, mouse, goat, and the like). The solution of conjugate is diluted with the dilution buffer (60 mM Tris-HCl buffer pH 7.8 containing 150 mM NaCl, 0.1% TWEEN™ 20 and 0.5% gelatine) and incubated for 1 hour at room temperature. Empty the plate and wash the wells with a series of 5 successive washes. Distribute 100 μl of a solution of substrate, such as for example para-nitrophenyl phosphate in the case where alkaline phosphatase is used as revealing enzyme. The pNPP is prepared in an amount of 1 mg/ml of reagent in 0.1M Tris-HCl buffer pH 9.5 containing 1.35M NaCl. Incubate the plate at 37° C. and the optical density of each of the wells is read at 405 nm after about 20 minutes. After recording the signals obtained, the optical density values are represented by means of a graph as a function of the logarithm of the serum dilution. The titre value is set as being equal to the dilution value for which 50% of the antigen-antibody complex is obtained.

### EXAMPLE 9

Immunoassay Method Specific for NSGP

The anti-peptide antibodies purified as described can be used to perform an immunoenzymatic assay of the extraction saturation or “sandwich” type in which the anti-peptide antibodies are absorbed in the wells of the microplate (they are the “capturing” antibodies). The NSGP present in the sample to be assayed is immunocaptured by the antibodies adsorbed and the presence of NSGP is revealed by a second labeled anti-peptide antibody. The assay can be carried out according to the following procedure: The anti-peptide antibodies used as tracer antibodies are obtained from purification methods from immunized rabbits.

The anti-peptide antibodies are bound to an insoluble support, which may be microtitre plate wells, polystyrene beads or any other material capable of adsorbing antibodies. A 96-well microplate is used for example. The primary antibody used to coat the tray is a rabbit polyclonal anti-peptide antibody directed against the N-terminal oligopeptide (#1) of NSGP. A stock solution of the antibody (5 mg/ml) in carbonate buffer pH 9.6 (Buffer A: carbonate-bicarbonate buffer, pH 9.6 consists of 1.59 g of Na₂CO₃, 2.93 g NaHCO₃ and made to 1 L with ddH₂O) is made and 100 μl per well (except blanks) is aliquotted and covered with parafilm and incubated overnight at 40° C. or alternatively 37° C. for 2 hours.

The antibody is aspirated away and immediately replaced with 150 μl blocking solution (Buffer A+1% BSA) to all wells, including blanks and incubated at 37° C. for at least 30 minutes. The samples are washed five times in ELISA wash solution pH 7.0 (6.055 g Tris Base, 29.22 g NaCl, 0.744 g EDTA and made to 1 L with ddH₂O and add 1 mL Triton X-100 then pH to 7.0 with 5M HCl). Purified NSGP is initially prepared in Antibody Buffer pH 7.0 (0.6055 g Tris Base, 2.922 g NaCl, 0.5 g BSA, 0.0372 g EDTA and made to 50 mL with ddH₂O, then add 50 μl Triton X-100 and finally pH to 7.0 with 5M HCl. 200 ng of the NSGP in solution is added in duplicate to the first wells and serial diluted down the plate for a standard curve (100, 50, 25, 12.5, 6, 3 1.5, 0.75 ng/well). For each sample neat serum is added to the first well and serially dilute down the plate in antibody buffer in triplicate, then incubated at 37° C. for 60 minutes. The tray is washed five times with ELISA wash solution. The addition of the second anti-peptide antibody raised against C-terminal oligopeptide (#2) from NSGP is blotinylated using standard procedures and 100 μl of a 1/1000 dilution to each well is added. The tray is incubated at 37° C. for 60 minutes, and the tray washed five times with ELISA wash solution and 100 μl of a 1/3000 dilution of Streptavidin alkaline phosphatase is added to each well. The tray is incubated in the dark for 20 minutes at room temperature. The wells are emptied, washed and then emptied before introducing 100 μl of a pNPP solution. The optical densities of the solutions contained in each of the wells are read at 405 nm with a 96-well microtitre plate reader. A volume of 50 μl of stop solution (1M sodium hydroxide) is added when the highest optical density reaches 2 to 2.5 units, that is to say after 20 to 30 minutes of incubation at 37° C.

The biological samples to be assayed are measured in the same manner and evaluated relative to the standard curve. If the spectrophotometric microplate reader comprises a suitable software for calculation, the representation can be made directly by the latter.

The method developed in the preceding example served as the basis for the development of a ready-for-use kit for assaying NSGP in any dissolved blood or tissue sample. This kit comprises all the following constituents: 1. 96-well immunotitre plate sensitized with the purified anti-peptide antibody. It can be a divisible plate (strips of 8, 16, 24 or 48 wells) or a non-divisible plate. In all cases, the strips or the plate are packaged in a blister in the presence of a dessicating agent for their preservation. 2. Solution for diluting the samples: buffered solution (Tris, phosphate) containing a protein (such as casein, BSA, ovalbumin) at a concentration of the order of 0.1% to 1%, an ionic or non-ionic detergent and a preserving agent (sodium azide or merthiolate). 3. Freeze-dried standard: this is human or recombinant NSGP and freeze-dried. 4. Concentrated washing buffer buffered solution (Tris, phosphate) containing NaCl, a detergent and a preserving agent such as those described above. 5. Solution of labeled tracer antibodies: this may be an anti-human NSGP monoclonal or polyclonal or peptide antibody obtained by immunization of animals with purified human NSGP or peptide derivatives thereof. These antibodies are labelled with biotin or coupled to a revealing enzyme (alkaline phosphatase or PAL, horseradish peroxidase or HRP). These antibodies are in solution in a mixture containing Tris or phosphate buffer, NaCl (150 mM), an overload protein (BSA, OVA, casein) at 0.1-1%, glycerol and a preserving agent. Alternatively, a third antibody against the second antibody may be labeled. 6. A solution of avidin coupled to a revealing enzyme: this solution is present only when the tracer antibody is biotinylated. Either avidin or streptavidin may be used. This protein may be coupled to PAL or HRP. It is in saline solution (Tris, phosphate) with an overload protein (BSA, OVA, casein) and a preserving agent adapted to the type of revealing (sodium azide proscribed for peroxidase). 7. Revealing substrate(s): pNPP in the case of alkaline phosphatase and ortho-phenylenediamine (OPD) in the case of peroxidase. 8. Substrate dilution buffer Basic buffer (Tris, ethanolamine) pH 9.5 containing magnesium chloride and a preserving agent (sodium azide or merthiolate) in the case of alkaline phosphatase. Acidic buffer (for example citrate pH 5.5) containing hydrogen peroxide (about 0.012%) and a preserving agent (merthiolate) in the case of peroxidase. 9. Stop solution: 1M sodium hydroxide solution containing a metal chelator such as 0.1M ethylenediaminetetraacetate (EDTA) in the case of alkaline phosphatase; 1M sulphuric acid solution in the case of revealing with peroxidase.

The implementation of the test is performed according to the following procedure: Collect venous blood in a glass tube without anticoagulant. Leave the blood to stand for a minimum of two hours before removing the serum. Centrifuge for 10 minutes at 2000×g and recover the serum. The serum samples are stable for 24 hours at 4° C. Beyond that, it is advisable to freeze the samples at −20° C. or better at −80° C. Dilution: the serum samples should be diluted in the solution for diluting the samples. A dilution value of between 1:40 and 1:80 is sufficient.

1. A method for diagnosing a disease state associated with oxidative stress in a subject, the method including the steps of:
measuring the level of non-selenium glutathione peroxidase protein in a biological fluid or tissue obtained from the subject over time to detect an increase in the level of non-selenium glutathione peroxidase protein in the subject; and/or
measuring the level of non-selenium glutathione peroxidase protein in a biological fluid or tissue obtained from the subject and comparing the measured level of non-selenium glutathione peroxidase protein with a control level.

2. The method of claim 1, wherein the method includes the steps of:
comparing the level of non-selenium glutathione peroxidase protein present in a first biological fluid sample to the level of non-selenium glutathione peroxidase protein present in a second biological fluid sample, wherein the first fluid is a fluid from a subject suspected of suffering from an increase in oxidative stress and the second fluid is a fluid from a subject that is not suffering from an increase in oxidative stress, wherein the first and second fluids are of the same fluid type, and wherein the non-selenium glutathione peroxidase protein is identified by an agent that specifically recognises the non-selenium glutathione peroxidase protein; and
categorizing the first sample as likely to be indicative of the onset of the disease state in the subject when the level of non-selenium glutathione peroxidase protein is found to be the higher in the first sample than in the second sample.

3. The method of claim 2 wherein the subject is human.

4. The method of claim 3 wherein the biological fluid that is assayed is selected from the list consisting of serum, plasma, whole blood, cerebro spinal fluid, amniotic fluid, and synovial fluid.

5. The method of claim 1 wherein the tissue that is assayed is epithelial, connective or muscle tissue.

6. The method of claim 4 wherein the agent that specifically recognises the non-selenium glutathione peroxidase protein is an antibody specific for non-selenium glutathione peroxidase.

7. The method of claim 6 wherein the disease state associated with oxidative stress is a neurodegenerative disease.

8. The method of claim 6 wherein the disease state associated with oxidative stress is Parkinson's disease.

9. The method of claim 6 wherein the disease state associated with oxidative stress is Alzheimer's disease.

10. The method of claim 6 wherein the disease state associated with oxidative stress is Dementia.

11. A method for detecting oxidative stress in a subject, the method including the steps of:
producing antibodies specific to at least one peptide fragment of non-selenium glutathione peroxidase protein, or derivative thereof,
obtaining a putative non-selenium glutathione peroxidase protein containing biological sample from the subject,
contacting the biological sample with the antibodies under conditions for formation of an antibody:non-selenium glutathione peroxidase protein complex, and
assaying for the formation of the antibody:non-selenium glutathione peroxidase protein complex to detect the presence and/or levels of non-selenium glutathione peroxidase protein.

12. The method of claim 11 wherein the step of assaying for the formation of antibody:non-selenium glutathione peroxidase protein complex involves detecting the complex using a second revealing antibody.

13. The method of claim 11 comprising the steps of binding specific anti-peptide antibodies to a support; immunocapture of non-selenium glutathione peroxidase protein in a sample of biological material by the antibodies; and
revealing of the immunocaptured non-selenium glutathione peroxidase protein by a second labelled anti-peptide antibody.

14. An antibody, or fragment thereof, that binds to an oligopeptide that includes the sequence of SEQ ID NO:1 or a fragment of SEQ ID NO:1, or the sequence of SEQ ID NO:2 or a fragment of SEQ ID NO:2.


SEQUENCE LISTING (5 sequences)

SEQ ID NO: 1 - protein, 20 aa, Artificial Sequence
  Synthetic oligopeptide corresponding to the human NSGP protein
RIRFHDFLGDSWGILFSHPR

SEQ ID NO: 2 - protein, 21 aa, Artificial Sequence
  Synthetic oligopeptide corresponding to the human NSGP protein
KKLFPKGVFTKELPSGKKYLR

SEQ ID NO: 3 - protein, 224 aa, Homo sapiens
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 4 - DNA, 29 bp, Artificial Sequence
  Synthetic PCR primer
ggcaattcatgcccggagggctgcttctc

SEQ ID NO: 5 - DNA, 33 bp, Artificial Sequence
  Synthetic PCR primer
ccgctcgagcgggttcccgcagacttaaggctg